(19) United States
(12) Plant Patent Application Publication
(10) Pub. No.: US 2015/0040275 P1
(43) Pub. Date: Feb. 5, 2015

(54) Prunus tree rootstock named 'Gi14813'

(50) ;

(71) Applicant: Consortium Deutsche Baumschulen GmbH, Ellerbek (DE)

(72) Inventor: Sabine Franken-Bembenek, Giessen (DE)

(73) Assignee: Consortium Deutsche Baumschulen GmbH, Ellerbek (DE)

(21) Appl. No.: 13/987,524

(22) Filed: Aug. 1, 2013

(51) Int. Cl. A01H 5/00

(52) U.S. Cl. PLT/180

(57) ABSTRACT

A new and distinct Prunus hybrid plant used as a rootstock for cherries that induces precocity and flat branching of the grafted plant.

### BOTANICAL CLASSIFICATION

Prunus hybrid

### VARIETAL DENOMINATION

‘Gi14813’

### BACKGROUND OF THE INVENTION

The present invention comprises a new and distinct cultivar of Prunus hybrid used as a rootstock known by the varietal name ‘Gi14813’. The new variety was discovered in Giessen, Germany in 1971. The new variety is the result of a planned breeding program between Prunus cerasus ‘Schattenmorelle’ (unpatented female parent) and a Prunus canescens (male parent). The new variety differs from its parents in that it has a triploid genome, whereas the female parent has a tetrapoloid genome and the male parent has a diploid genome. The purpose of the breeding program was to produce a series of size-controlling, productive, and precocious rootstocks for sweet cherries. The new variety has been trial and field tested and has been found to retain its distinctive characteristics and remain true to type through successive propagations.

The new variety is similar to prunus rootstock ‘GI 148/2’ (U.S. Plant Pat. No. 9,622) in its rootstock induction of precosity, rootstock induction of flat branching, and the absence of root suckers. The color of the leaves of emerging branches in the Spring is more reddish (anthocyanin coloration) than ‘GI 148/2’. Further, the vigor of a grafted tree on the new variety is greater than on ‘GI 148/2’.

### DESCRIPTION OF THE DRAWINGS

The accompanying photographic drawings illustrate the new cultivar, with the color being as nearly true as is possible with color illustrations of this type:

FIG. 1 is a close-up picture of the leaves and branches of the new variety;

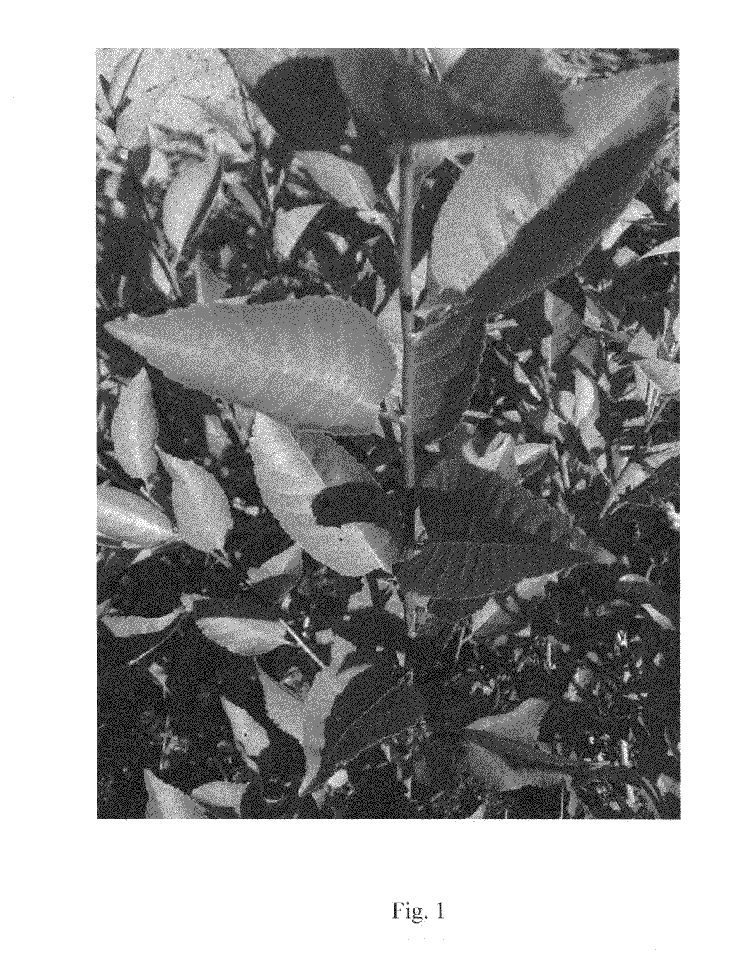

FIG. 2 is a close-up picture that shows both surfaces of the leaves of the new variety;

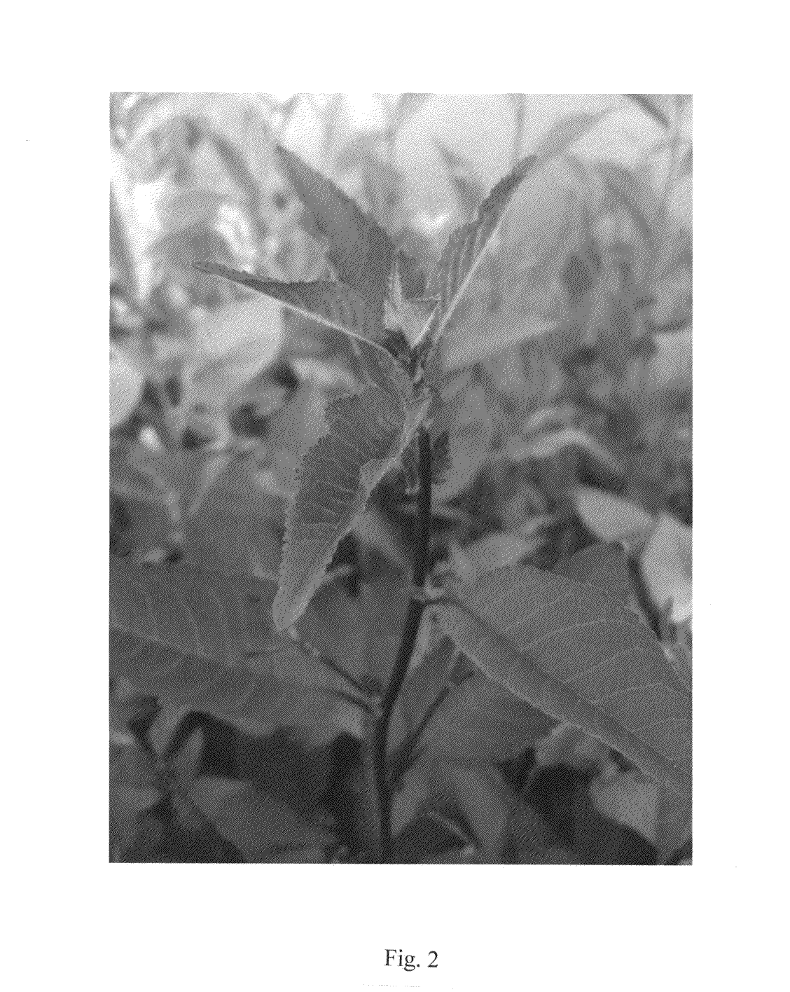

FIG. 3 is a picture of a field of plants of the new variety; and

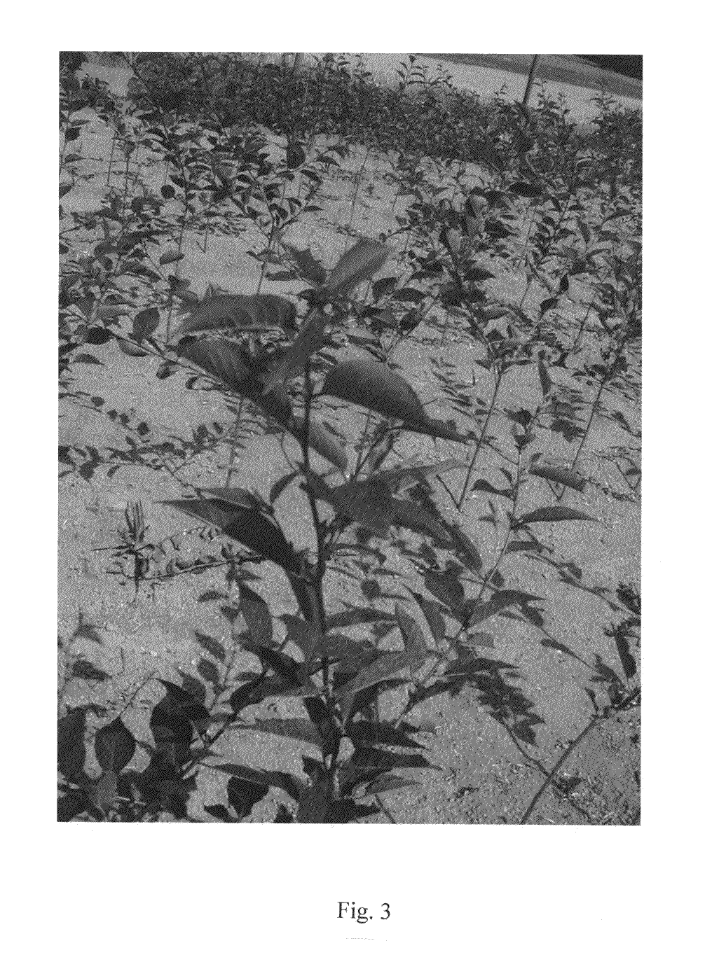

FIG. 4 is a further picture of a field of plants of the new variety.

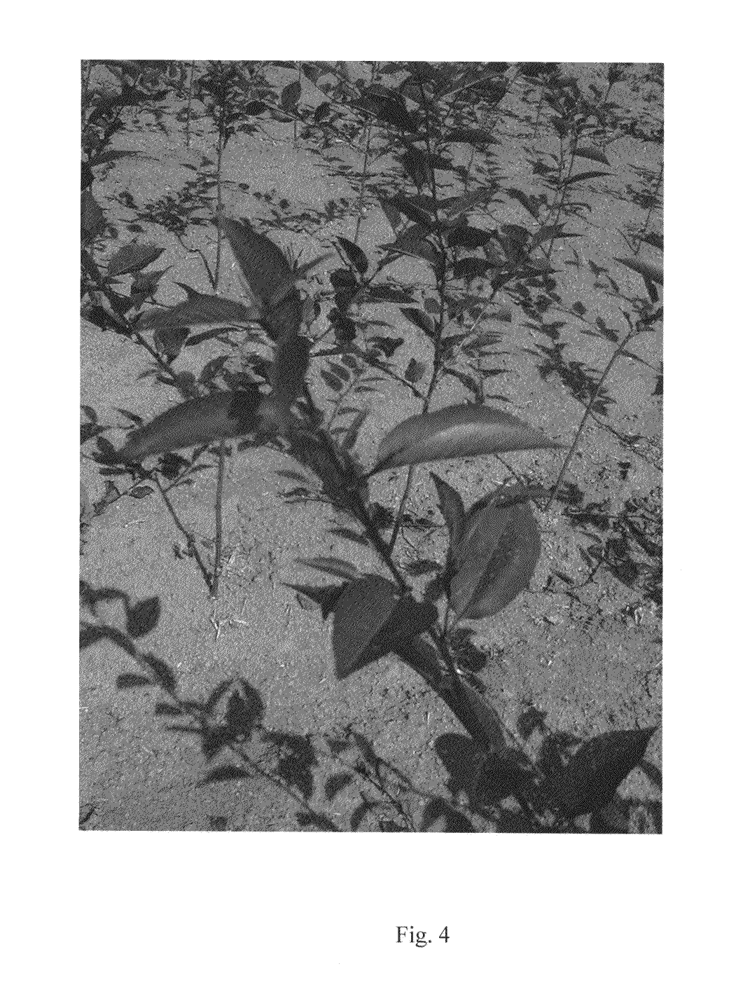

### DESCRIPTION OF THE PLANT

The following detailed description sets forth the characteristics of the new cultivar. The data which defines these characteristics were collected by asexual reproduction carried by green cuttings under mist and in vitro conditions. The first asexual reproduction occurred in approximately 1975 in Giessen, Germany. The color readings were taken in natural daylight. Color references are primarily to the R.H.S. Colour Chart of The Royal Horticultural Society of London.
Plant:
   Use.—As a rootstock for sweet, sour, and ornamental cherries.
   Branch color.—144A.
   Leaves.—Length: Medium. Width: Medium. Form: Ovate. Apex: Acute. Base: Obtuse. Margin: Serrate. Pubescence: Weak presence on lower surface. Color: Upper surface: 137A. Lower surface: 137C. Petiole length: 1.0 cm.
   Flowers.—Pedicel length: 1.2 cm. Bud color: 56D/69C. Bloom timing: Late, similar to the cultivar ‘Regina’. Petals: Number: Five. Length: 0.9 cm. Shape: Round. Color (fully opened): White on both surfaces. Sepals: Length: 0.4 cm. Color: 144A.
   Tree winter hardiness.—Good.
   Performance as a grafted rootstock.—Root sprouts: No suckers observed. Anchorage: Good. Compatibility: Good. Vigor: Reduces size of tree to about 60% of a Prunus avium rootstock, comparable to the vigor of Gisela 6 and is somewhat more vigorous than Gisela 5.

I claim:

1. A new and distinct variety of Prunus hybrid plant used as rootstock as shown and described herein.
